(19) United States
(12) Patent Application Publication
(10) Pub. No.: US 2007/0148211 A1
(43) Pub. Date: Jun. 28, 2007

(54) PROCESSES FOR MAKING PARTICLE-BASED PHARMACEUTICAL FORMULATIONS FOR ORAL ADMINISTRATION

(75) Inventors: David Altreuter, Wayland, MA (US); Howard Bernstein, Cambridge, MA (US); Luis A. Brito, Winchester, MA (US); Shaina Brito, Winchester, MA (US); Donald E. Chickering III, Framingham, MA (US); Eric K. Huang, Cambridge, MA (US); Rajeev A. Jain, Framingham, MA (US); Sridhar Narasimhan, Elgin, IL (US); Julie A. Straub, Winchester, MA (US)

Correspondence Address: SUTHERLAND ASBILL & BRENNAN LLP, 999 PEACHTREE STREET, N.E., ATLANTA, GA, 30309, US

(73) Assignee: Acusphere, Inc., Watertown, MA (US)

(21) Appl. No.: 11/610,802

(22) Filed: Dec. 14, 2006

Related U.S. Application Data
(60) Provisional application No. 60/750,750, filed on Dec. 15, 2005.

(51) Int. Cl. A61K 9/28; A61K 9/48; A61K 9/20; A61K 9/14

(52) U.S. Cl. 424/441; 424/451; 424/464; 424/489

(57) ABSTRACT

A method is provided for making an oral dosage form of a pharmaceutical agent which includes the steps of (a) providing particles which include a pharmaceutical agent; (b) blending the particles with particles of a pre-processed excipient to form a primary blend, wherein the pre-processed excipient is prepared by (i) dissolving a bulking agent (e.g., a sugar) and at least one non-friable excipient (e.g., a waxy or liquid surfactant) in a solvent to form an excipient solution, and (ii) removing the solvent from the excipient solution to form the pre-processed excipient in dry powder form; (c) milling the primary blend to form a milled pharmaceutical formulation blend that includes microparticles or nanoparticles of the pharmaceutical agent; and (d) processing the milled pharmaceutical formulation blend into a solid oral dosage form or liquid suspension for oral administration. The process yields formulations having improved wettability or dispersibility.

### CROSS-REFERENCE TO RELATED APPLICATIONS

This application claims benefit of U.S. Provisional Application No. 60/750,750, filed Dec. 15, 2005. The application is incorporated herein by reference.

### BACKGROUND OF THE INVENTION

This invention is generally in the field of pharmaceutical compositions comprising particles, such as microparticles, and more particularly to methods for making particulate blend formulations for oral administration.

Microparticles comprising therapeutic and diagnostic agents are known to be useful for enhancing the controlled delivery of such agents to humans or animals. For these applications, microparticles having very specific sizes and size ranges are needed in order to effectively deliver these agents. Many drug formulations are produced in a dry powder form for use in one or more particular dosage forms.

Oral dosage forms of therapeutic microparticles require that the microparticles disperse in vivo in the oral cavity (e.g., orally disintegrating tablets) or in the gastro-intestinal tract for dissolution and subsequent bioavailability of the therapeutic agent (e.g., tablet, capsule, or suspension). Microparticles, particularly those consisting of hydrophobic pharmaceutical agents, tend to be poorly dispersible in aqueous media. This may undesirably alter the microparticle formulation's performance and/or reproducibility. Dispersibility depends on a variety of factors, including the materials and methods used in making the microparticles, the surface (i.e., chemical and physical) properties of the microparticles, the temperature of the suspending medium or vehicle, and the humidity and compaction forces to which the microparticles are exposed in the case of oral dosage forms. It would therefore be useful to provide a process that creates well dispersing microparticle formulations. Such a process should be simple and operate at conditions to minimize equipment and operating costs and to avoid degradation of the pharmaceutical agent.

Excipients often are added to the microparticles and pharmaceutical agents in order to provide the microparticle formulations with certain desirable properties or to enhance processing of the microparticle formulations. For example, the excipients can facilitate administration of the microparticles, minimize microparticle agglomeration upon storage or upon reconstitution, facilitate appropriate release or retention of the active agent, and/or enhance shelf life of the product. Representative types of these excipients include osmotic agents, bulking agents, surfactants, preservatives, wetting agents, pharmaceutically acceptable carriers, diluents, binders, disintegrants, glidants, and lubricants. It is important that the process of combining these excipients and microparticles yield a uniform blend. Combining these excipients with the microparticles can complicate production and scale-up; it is not a trivial matter to make such microparticle pharmaceutical formulations, particularly on a commercial scale.

Furthermore, certain desirable excipient materials are difficult to mill or blend with pharmaceutical agent microparticles. For example, excipients characterized as liquid, waxy, non-crystalline, or non-friable are not readily blended uniformly with drug containing particles and/or are not readily processed through a mill. Conventional dry blending of such materials may not yield the uniform, intimate mixtures of the components, which pharmaceutical formulations require. For example, dry powder formulations therefore should not be susceptible to batch-to-batch or intra-batch compositional variations. Rather, production processes for a pharmaceutical formulation must yield consistent and accurate dosage forms. Such consistency in a dry powder formulation may be difficult to achieve with an excipient that is not readily blended or milled. It therefore would be desirable to provide methods for making uniform blends of microparticles and difficult to blend excipients. Such methods desirably would be adaptable for efficient, commercial scale production.

It therefore would be desirable to provide improved methods for making blended particle or microparticle pharmaceutical formulations and solid oral dosage forms that have high content uniformity and that disperse well upon oral administration. In addition, it would be desirable to provide a solid oral dosage form of a drug, particularly a poorly water soluble drug, that has improved wettability.

### SUMMARY OF THE INVENTION

Methods are provided for making a pharmaceutical particle blend formulation for oral administration. In one embodiment, the method includes the steps of (a) providing particles which comprise a pharmaceutical agent; (b) blending the particles with particles of a pre-processed excipient to form a primary blend, wherein the pre-processed excipient is prepared by (i) dissolving a bulking agent and at least one non-friable excipient in a solvent to form an excipient solution, and (ii) removing the solvent from the excipient solution to form the pre-processed excipient in dry powder form; (c) milling the primary blend to form a milled pharmaceutical formulation blend, which comprises microparticles or nanoparticles of the pharmaceutical agent; and (d) processing the milled pharmaceutical formulation blend into a solid oral dosage form or liquid suspension for oral administration. In a preferred embodiment, the milled pharmaceutical formulation blend is processed into a solid oral dosage form selected from tablets, capsules, orally disintegrating wafers, and sprinkle packets. In one embodiment, the milling step includes jet milling. In various embodiments, the step of removing the solvent may include spray drying, lyophilization, vacuum drying, or freeze drying. In one embodiment, the pre-processed excipient particles are milled before blending with the particles of step (a).

The particles of step (a) may be microparticles. In various embodiments, the bulking agent comprises at least one sugar, sugar alcohol, starch, amino acid, or combination thereof. Examples of bulking agents include lactose, sucrose, maltose, mannitol, sorbitol, trehalose, galactose, xylitol, erythritol, and combinations thereof. The non-friable excipient may be a liquid, waxy, or non-crystalline compound, In a preferred embodiment, the non-friable excipient comprises a surfactant, such as a waxy or liquid surfactant. Examples of possible surfactants include docusate sodium or a polysorbate. In one embodiment, the pharmaceutical agent has a solubility in water of less than 10 mg/mL at 25° C. In various embodiments, the microparticles or nanoparticles of pharmaceutical agent in the milled pharmaceutical formulation blend have a volume average diameter of less than 100 μm. For instance, the volume average diameter may be less than 20 μm, preferably less than 10 μm.

In a particular embodiment, the method includes the steps of (a) providing particles which comprise a pharmaceutical agent; (b) blending the particles with particles of a pre-processed excipient to form a primary blend, wherein the pre-processed excipient is prepared by (i) dissolving a bulking agent and at least one non-friable surfactant in a solvent to form an excipient solution, wherein the bulking agent comprises at least one sugar, sugar alcohol, starch, amino acid, or combination thereof, and (ii) removing the solvent from the excipient solution to form the pre-processed excipient in dry powder form; (c) jet milling the primary blend to form a milled pharmaceutical formulation blend, which comprises microparticles or nanoparticles of the pharmaceutical agent; and (d) processing the milled pharmaceutical formulation blend into a solid oral dosage form or liquid suspension for oral administration.

In another embodiment, a method is provided for making a solid oral dosage form of a pharmaceutical agent that includes the steps of (a) providing particles which comprise a pharmaceutical agent; (b) blending the particles which comprise a pharmaceutical agent with particles of an excipient to form a first blend; (c) milling the first blend to form a second blend, which comprises microparticles or nanoparticles of the pharmaceutical agent; (d) granulating the second blend to form a granulated milled blend; and (e) processing the granulated milled blend into an oral dosage form, In one embodiment, the milling step includes jet milling. In various embodiments, the granulated milled blend is processed into a solid oral dosage form selected from the group consisting of tablets, capsules, orally disintegrating wafers, and sprinkle packets. Step (e) may include blending the granulated milled blend with at least one sugar and at least one disintegrant to form a third blend, and then tabletting the third blend to form an orally disintegrating wafer. In an alternative embodiment, the granulated milled blend may be processed into a liquid suspension for oral administration. In one embodiment, the pharmaceutical agent has a solubility in water of less than 10 mg/mL at 25° C. In one embodiment, the particles of step (a) are microparticles.

In another aspect, a method is provided for making a solid oral dosage form of a pharmaceutical agent that includes the steps of (a) providing particles which comprise a pharmaceutical agent; (b) blending the particles of pharmaceutical agent with particles of at least one excipient to form a first blend; (c) milling the first blend to form a milled blend which comprises microparticles; and (d) processing the milled blend into a solid oral dosage form, wherein the size of the microparticles following reconstitution of the solid oral dosage form is not more than 300%, preferably not more than 150%, of the size of the microparticles in the milled blend pre-processing. In one embodiment, step (d) includes compacting the milled blend into a unitary dosage form selected from tablets and orally disintegrating wafers. In one embodiment, the milling of step (c) includes jet milling. In one embodiment, the pharmaceutical agent has a solubility in water of less than 10 mg/mL at 25° C. In one embodiment, the microparticles of pharmaceutical agent in the milled blend have a volume average diameter of less than 100 μm. For instance, the volume average diameter may be less than 10 μm.

In another aspect, a method is provided for using a non-friable excipient in a dry powder process for making a pharmaceutical blend formulation for oral administration. In one embodiment, the method includes the steps of (a) providing particles which comprise a pharmaceutical agent; (b) blending the particles with particles of a pre-processed excipient to form a primary blend, wherein the pre-processed excipient is prepared by (i) dissolving a bulking agent and at least one non-friable excipient in a solvent to form an excipient solution, and (ii) removing the solvent from the excipient solution to form the pre-processed excipient in dry powder form; and (c) milling the primary blend to form a milled pharmaceutical formulation blend, which comprises microparticles or nanoparticles of the pharmaceutical agent. In one case, the milling includes jet milling. In various embodiments, the step of removing the solvent comprises spray drying, lyophilization, vacuum drying, or freeze drying. In preferred embodiments, the bulking agent includes at least one sugar, sugar alcohol, starch, amino acid, or combination thereof. The non-friable excipient may be a liquid, waxy, or non-crystalline compound. In one embodiment, the pharmaceutical agent has a solubility in water of less than 10 mg/mL at 25° C. The microparticles or nanoparticles of pharmaceutical agent in the milled pharmaceutical formulation blend may have a volume average diameter of less than 10 μm.

In another aspect, pharmaceutical formulations made by the foregoing methods are provided. In one embodiment, an oral disintegrating tablet pharmaceutical formulation is provided that includes a mixture of granules formed by granulation of a milled blend of (i) microparticles which comprise a pharmaceutical agent, and (ii) excipient particles; particles of at least one sugar; and particles of at least one disintegrant, wherein the mixture has been compressed into a tablet or wafer form. In another embodiment, a solid oral dosage form of a pharmaceutical agent is provided that includes a milled blend of microparticles of a pharmaceutical agent blended and particles of at least one excipient, which milled blend has been processed into a solid oral dosage form, wherein the size of the microparticles following reconstitution of the solid oral dosage form is not more than 300%, preferably not more than 200%, of the size of the microparticles in the milled blend pre-processing.

### BRIEF DESCRIPTION OF THE DRAWINGS

FIG. 1 is a process flow diagram of one embodiment of a process for making an oral dosage form of a pharmaceutical formulation which includes a milled dry powder blend of a drug and a pre-processed excipient as described herein.

FIG. 2 is a process flow diagram of one embodiment of a process for making an oral dosage form of a pharmaceutical formulation which includes a milled and granulated dry powder blend of a drug and an excipient as described herein.

FIG. 3 is a process flow diagram of one embodiment of a process for making a tablet or orally disintegrating wafer form of a pharmaceutical formulation which includes a jet milled dry powder blend of a drug-containing microparticles and excipient particles as described herein.

FIG. 4 is a process flow diagram of one embodiment of a process for pre-processing a non-friable excipient into a dry powder form.

FIGS. 5A-C are light microscope images of microparticles taken before blending, after blending, and after blending followed by jet milling.

FIGS. 6A-B are light microscope images of celecoxib particles reconstituted from a jet milled blend of celecoxib and non-pre-processed excipients.

FIGS. 7A-B are light microscope images of celecoxib particles reconstituted from a jet milled blend of celecoxib and pre-processed excipients.

FIGS. 8A-B are light microscope images of reconstituted celecoxib from a blend of excipient particles and celecoxib particles.

FIGS. 9A-B are light microscope images of reconstituted celecoxib from a blend of excipient particles and milled celecoxib particles.

FIGS. 10A-B are light microscope images of reconstituted celecoxib from a jet milled blend of excipient particles and celecoxib particles.

FIGS. 11A-C are scanning electron microscopy (SEM) images, and FIGS. 11D-J are Energy Dispersive X-Ray Spectroscopy (EDS) images with analysis for chlorine or sodium, of dry powder pharmaceutical formulation blends made by different processes described herein.

### DETAILED DESCRIPTION OF THE INVENTION

Improved processing methods have been developed for making an oral dosage form of a pharmaceutical formulation that includes a uniform blend of pharmaceutical agent particles and excipient particles. It has been determined that better dispersibility or wettability of the formulations may be obtained by the ordered steps of blending particles of pharmaceutical agent with an excipient and then milling the resulting blend, as compared to blends prepared without this combination of steps. It has also been beneficially discovered that certain useful but difficult-to-mill excipient materials can be used in the process if they are themselves first subjected to a“pre-processing” treatment that transforms the liquid, waxy, or otherwise non-friable excipient into a dry powder form that is suitable for blending and milling in a dry powder form. By milling after blending, it was found that the dry powder blend advantageously has decreased pharmaceutical agent particle-to-pharmaceutical agent particle contact in the dry state, thereby providing a blend that is more readily or more rapidly wettable and dispersible. By post milling the blend, the particles comprising pharmaceutical agents come into intimate contact with excipient particles, such as mannitol in the powder blend (matrix), and are rapidly wetted on contact with water. Thus, a suspension having an increased amount of discrete particles comprising pharmaceutical agent is produced.

The presence of other excipients like polymers and surfactants (in the powder blend or the resultant suspension) provides supplementary stability forces (steric and electrostatic interaction) to the dispersed particles comprising pharmaceutical agent. In addition, during milling of the blend of excipient particles and particles comprising pharmaceutical agent, there is the potential for reduction in the size of the excipient particles. Such a reduction in particle size of the excipient particles would potentially lead to more rapid dissolution of the excipient particles. Thus, reconstitution of drug particles from the dosage form in the oral cavity or GI tract would, it is theorized, be improved.

As used herein, the term “dispersibility” includes the suspendability of a powder (e.g., a quantity or dose of microparticles) within a liquid. Accordingly, the term “improved dispersibility” refers to a reduction of particle-particle interactions of the microparticles of a powder within a liquid. In addition, the microparticles as processed herein can be further formulated into solid oral dosage forms having improved disintegration properties. As used herein, “improved disintegration properties” refers to improvements in dosage form disintegration time and/or improvements in the dispersibility of the suspension that results from the disintegration of the solid oral dosage form. Dosage form disintegration time can be evaluated using the USP method for disintegration, or using a visual evaluation for time to tablet disintegration within an aqueous media where disintegration is considered complete when tablet fragments are no larger than 1 mm. Improvements in dispersibility can be evaluated using methods that examine the increase in concentration of suspended particles or a decrease in the concentration or size of agglomerates. These methods include visual evaluation for turbidity of the suspension, direct turbidity analysis using a turbidimeter or a visible spectrophotometer, light microscopy for evaluation of concentration of suspended particles and/or concentration of agglomerated particles, Coulter counter analysis for particle concentration or particle size in suspension, or light scattering methods of analysis for particle size in suspension. An increase in turbidity, an increase in the concentration of suspended particles, a decrease in agglomerated particles, or a decrease in the particle size in suspension based on a volume mean indicates an improvement in dispersibility. Improvements in dispersibility can also be assessed as an increase in wettability of the powder using contact angle measurements.

The pharmaceutical formulations made as described herein are intended to be administered to a patient (i.e., human or animal in need of the pharmaceutical agent) to deliver an effective amount of a therapeutic, diagnostic or prophylactic agent.

As used herein, the terms “comprise,” “comprising,” “include,” and “including” are intended to be open, non-limiting terms, unless the contrary is expressly indicated.

The Methods

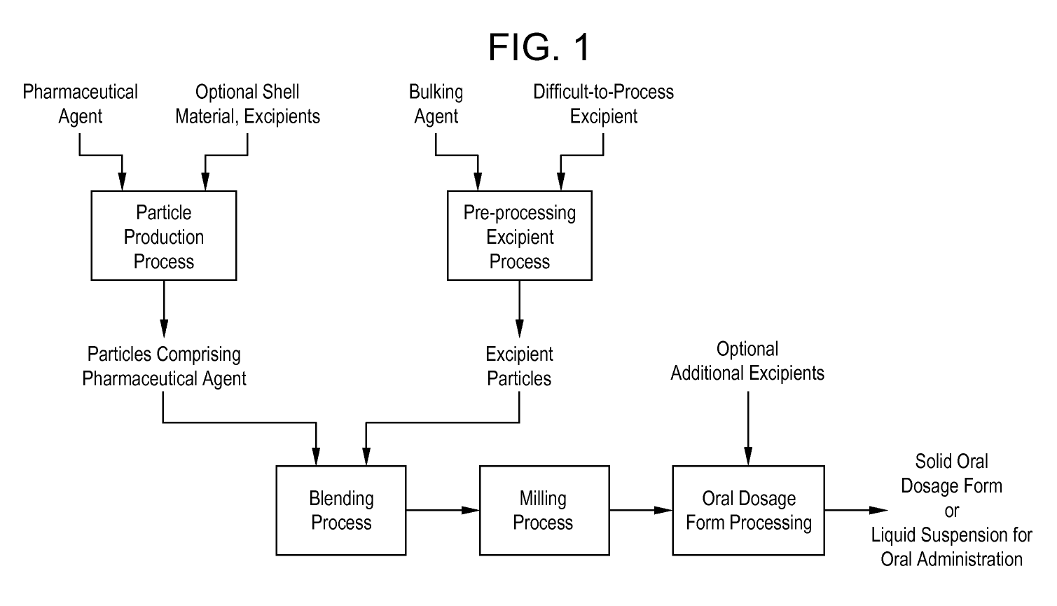

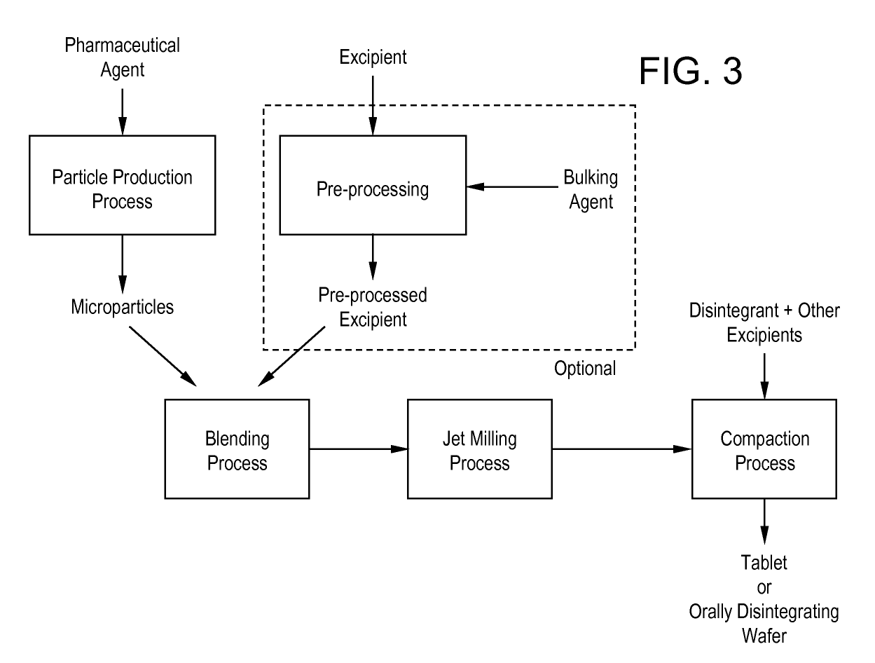

In one embodiment, the method for making an oral dosage form of a pharmaceutical agent includes the steps of (a) providing particles which comprise a pharmaceutical agent; (b) blending the particles with particles of a pre-processed excipient to form a primary blend, wherein the pre-processed excipient is prepared by (i) dissolving a bulking agent and at least one non-friable excipient in a solvent to form an excipient solution, and (ii) removing the solvent from the excipient solution to form the pre-processed excipient in dry powder form; (c) milling the primary blend to form a milled pharmaceutical formulation blend, which comprises microparticles or nanoparticles of the pharmaceutical agent; and (d) processing the milled pharmaceutical formulation blend into a solid oral dosage form or liquid suspension for oral administration. See FIG. 1 and FIG. 3. In a more general form, the method can be seen as one for making a particle-based pharmaceutical formulation comprising the steps of: (a) providing particles which comprise a pharmaceutical agent; (b) blending the particles with particles of a pre-processed excipient to form a primary blend, wherein the pre-processed excipient is prepared by (i) dissolving a bulking agent and at least one non-friable excipient in a solvent to form an excipient solution, and (ii) removing the solvent from the excipient solution to form the pre-processed excipient in dry powder form; (c) milling the primary blend to form a milled pharmaceutical formulation blend, which comprises microparticles or nanoparticles of the pharmaceutical agent.

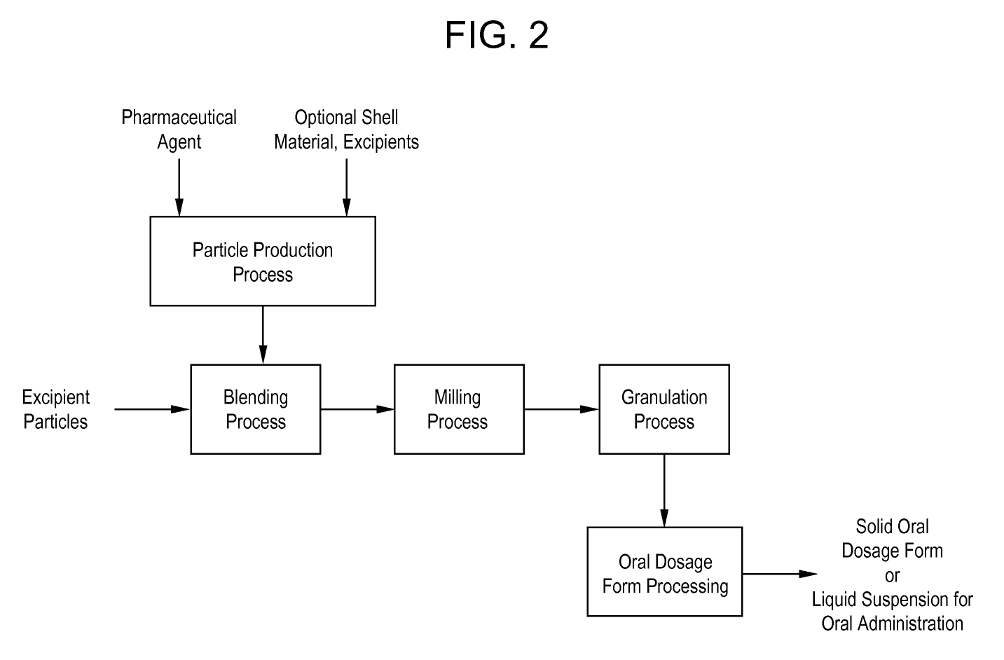

In another embodiment, the method for making a oral dosage form of a pharmaceutical agent includes the steps of (a) providing particles which comprise a pharmaceutical agent; (b) blending the particles which comprise a pharmaceutical agent with particles of an excipient to form a first blend; (a) milling the first blend to form a second blend, which comprises microparticles or nanoparticles of the pharmaceutical agent; (d) granulating the second blend to form a granulated milled blend; and (e) processing the granulated milled blend into an oral dosage form. See FIG. 2. In one particular embodiment, step (e) includes the sub-steps of blending the granulated milled blend with at least one sugar and at least one disintegrant to form a third blend, and tabletting the third blend to form an orally disintegrating wafer. In one embodiment, the combination of jet milling and granulation are believed to be particularly advantageous in the production of an orally disintegrating tablet (in particular for poorly water soluble drugs). An oral disintegrating tablet made by such a combination of steps has been observed to exhibit excellent wettability, to give both good reconstitution and favorable disintegration times. In another example, the Granulated milled blend is processed into tablets, capsules, or sprinkle packets. In still another example, the granulated milled blend is processed into a liquid suspension for oral administration.

In another embodiment, a method is provided for making a solid oral dosage form of a pharmaceutical agent. In a preferred embodiment, the method includes the steps of (a) providing particles which comprise a pharmaceutical agent; (b) blending the particles of pharmaceutical agent with particles of at least one excipient to form a first blend; (c) milling the first blend to form a milled blend which comprises microparticles; and (d) processing the milled blend into a solid oral dosage form, wherein the size of the microparticles following reconstitution of the solid oral dosage form is no more than 300%, preferably no more than 200%, and more preferably no more than 150%, of the size of the microparticles in the milled blend pre-processing. In one particular embodiment, step (d) includes compacting the milled blend into a unitary dosage form selected from tablets and orally disintegrating wafers.

The processes described herein generally can be conducted using batch, continuous, or semi-batch methods. These processes described herein optionally may further include separately milling some or all of the components (e.g., pharmaceutical agent particles, excipient particles) of the blended formulation before they are blended together. In preferred embodiments, the excipient and pharmaceutical agent are in a dry powder form.

Particle Production

The skilled artisan can envision many ways of making particles useful for the methods and formulations described herein, and the following examples describing how particles may be formed or provided are not intended to limit in any way the methods and formulations described and claimed herein. The particles comprising pharmaceutical agent that are used or included in the methods and formulations described herein can be made using a variety of techniques known in the art. Suitable techniques may include solvent precipitation, crystallization, spray drying, melt extrusion, compression molding, fluid bed drying, solvent extraction, hot melt encapsulation, phase inversion encapsulation, and solvent evaporation.

For instance, the microparticles may be produced by crystallization. Methods of crystallization include crystal formation upon evaporation of a saturated solution of the pharmaceutical agent, cooling of a hot saturated solution of the pharmaceutical agent addition of antisolvent to a solution of the pharmaceutical agent (drowning or solvent precipitation), pressurization, addition of a nucleation agent such as a crystal to a saturated solution of the pharmaceutical agent, and contact crystallization (nucleation initiated by contact between the solution of the pharmaceutical agent and another item such as a blade).

Another way to form the particles, preferably microparticles, is by spray drying. See, e.g., U.S. Pat. No. 5,853,698 to Straub et al.; U.S. Pat. No. 5,611,344 to Bernstein et al.; U.S. Pat. No. 6,395,300 to Straub et al.; and U.S. Pat. No. 6,223,455 to Chickering III, et at., which are incorporated herein by reference. As defined herein, the process of “spray drying” a solution containing a pharmaceutical agent and/or shell material refers to a process wherein the solution is atomized to form a fine mist and dried by direct contact with hot carrier gases. Using spray drying equipment available in the art, the solution containing the pharmaceutical agent and/or shell material may be atomized into a drying chamber, dried within the chamber, and then collected via a cyclone at the outlet of the chamber. Representative examples of types of suitable atomization devices include ultrasonic, pressure feed, air atomizing, and rotating disk. The temperature may be varied depending on the solvent or materials used. The temperature of the inlet and outlet ports can be controlled to produce the desired products. The size of the particulates of pharmaceutical agent and/or shell material is a function of the nozzle used to spray the solution of pharmaceutical agent and/or shell material, nozzle pressure, the solution and atomization flow rates, the pharmaceutical agent and/or shell material used, the concentration of the pharmaceutical agent and/or shell material, the type of solvent, the temperature of spraying (both inlet and outlet temperature), and the molecular weight of a shell material such as a polymer or other matrix material.

A further way to make the particles is through the use of solvent evaporation, such as described by Mathiowitz, et al., J. Scanning Microscopy, 4:329 (1990); Beck, et al., Fertil. Steril, 31:545 (1979) and Benita, et al., J. Pharm. Sci., 73:1721 (1984). In still another example, hot-melt microencapsulation may be used, such as described in Mathiowitz, et al., Reactive Polymers, 6:275 (1987). In another example, phase inversion encapsulation may be used, such as described in U.S. Pat. No. 6,143,211 to Mathiowitz, et al. This causes a phase inversion and spontaneous formation of discrete microparticles, typically having an average particle size of between 10 nm and 10 μm.

In yet another approach, a solvent removal technique may be used, wherein a solid or liquid pharmaceutical agent is dispersed or dissolved in a solution of a shell material in a volatile organic solvent and the mixture is suspended by stirring in an organic oil to form an emulsion. Unlike solvent evaporation, however, this method can be used to make microparticles from shell materials such as polymers with high melting points and different molecular weights. The external morphology of particles produced with this technique is highly dependent on the type of shell material used.

In another approach, an extrusion technique may be used to make microparticles of shell materials by dissolving the shell material (e.g., gel-type polymers, such as polyphosphazene or polymethylmethacrylate) in an aqueous solution, and extruding the material through a microdroplet forming device, producing microdroplets that fall into a slowly stirred hardening bath of an oppositely charged ion or polyelectrolyte solution.

Pre-Processing the Excipient

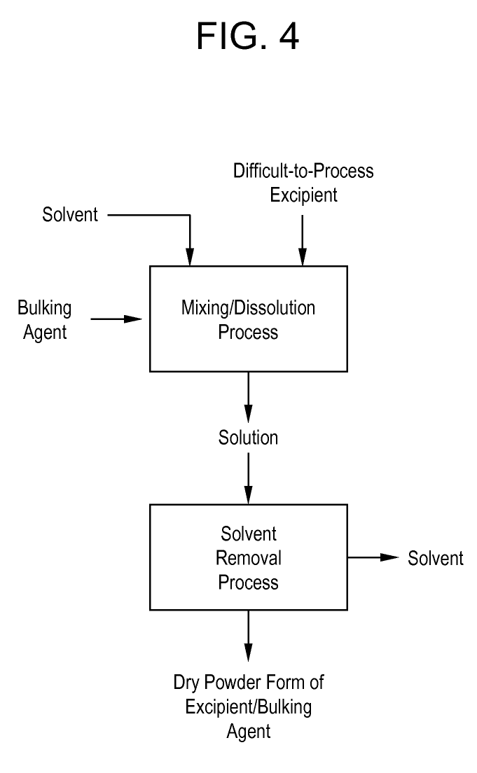

When it is necessary or desirable to convert a liquid, waxy, or otherwise non-friable excipient into a dry powder form suitable for blending and milling, these difficult-to-mill and difficult-to-blend excipient materials are “pre-processed.” In preferred embodiments, the pre-processed excipient that is used or included in the methods and formulations described herein is prepared by (i) dissolving a bulking agent and at least one non-friable excipient in a solvent to form an excipient solution, and then (ii) removing the solvent from the excipient solution to form the pre-processed excipient in dry powder form. See FIG. 4. The dissolution of bulking agent and at least one non-friable excipient in a solvent can be done simply by mixing appropriate amounts of these three components together in any order to form a well mixed solution. A variety of suitable methods of solvent removal known in the art may be used in this process. In one embodiment, the step of removing the solvent comprises spray drying. In another embodiment, the step of removing the solvent comprises lyophilization, vacuum drying, or freeze drying. The pre-processed excipient in dry powder form optionally may be milled prior to blending with the particles comprising pharmaceutical agent.

It is contemplated that the particles of pharmaceutical agent can be blended with one or more pre-processed excipients, and optionally, can be combined with one or more excipients that have not been pre-processed. The pharmaceutical agent particles can be blended with pre-processed excipient(s) either before or after blending with excipient(s) that have not been pre-processed. One or more of the excipients may be milled prior to combining with the pharmaceutical agent particles.

Blending and Milling

The particles of pharmaceutical agent are blended with one or more other excipient particulate materials, in one or more steps, and then the resulting blend is milled. Content uniformity of solid-solid pharmaceutical blends is critical. Comparative studies indicate that the milling of a blend (drug plus excipient) can yield a dry powder pharmaceutical formulation that exhibits improved wettability and/or dispersibility as compared to a formulation made by milling and then blending or by blending without milling. That is, the sequence of the two steps is important to the performance of the ultimate oral dosage form. In a preferred embodiment, pharmaceutical agent microparticles are blended with one or more excipients of interest, and the resulting blend is then jet milled to yield a uniform mixture of microparticles and excipient.

1. Blending

The skilled artisan can envision many ways of blending particles in and for the methods and formulations described herein, and the following examples describing how particles may be blended are not intended to limit in any way the methods and formulations described and claimed herein. The blending can be conducted in one or more steps, in a continuous, batch, or semi-batch process. For example, if two or more excipients are used, they can be blended together before, or at the same time as, being blended with the pharmaceutical agent microparticles.

The blending can be carried out using essentially any technique or device suitable for combining the microparticles with one or more other materials (e.g., excipients) effective to achieve uniformity of blend. The blending process may be performed using a variety of blenders. Representative examples of suitable blenders include V-blenders, slant-cone blenders, cube blenders, bin blenders, static continuous blenders, dynamic continuous blenders, orbital screw blenders, planetary blenders, Forberg blenders, horizontal double-arm blenders, horizontal high intensity mixers, vertical high intensity mixers, stirring vane mixers, twin cone mixers, drum mixers, and tumble blenders. The blender preferably is of a strict sanitary design required for pharmaceutical products.

Tumble blenders are often preferred for batch operation. In one embodiment, blending is accomplished by aseptically combining two or more components (which can include both dry components and small portions of liquid components) in a suitable container. One example of a tumble blender is the TURBULA™, distributed by Glen Mills Inc., Clifton, N.J., USA, and made by Willy A. Bachofen A G, Maschinenfabrik, Basel, Switzerland.

For continuous or semi-continuous operation, the blender optionally may be provided with a rotary feeder, screw conveyor, or other feeder mechanism for controlled introduction of one or more of the dry powder components into the blender.

2. Milling

The milling step is used to fracture and/or deagglomerate the blended particles to achieve a desired particle size and size distribution, as well as to enhance distribution of the particles within the blend. The skilled artisan can envision many ways of milling particles or blends in the methods and formulations described herein, and the following examples describing how such particles or blend may be milled are not intended to limit in any way the methods and formulations described and claimed herein. A variety of milling processes and equipment known in the art may be used. Examples include hammer mills, ball mills, roller mills, disc grinders and the like. Preferably, a dry milling process is used.

In a preferred technique, the milling comprises jet milling. Jet milling is described for example in U.S. Pat. No. 6,962,006 to Chickering III et al., which is incorporated herein by reference. As used herein, the terms “jet mill” and “jet milling” include and refer to the use of any type of fluid energy impact mills, including spiral jet mills, loop jet mills, and fluidized bed jet mills, with or without internal air classifiers. In one embodiment the jet milling process conditions are selected so that the size and morphology of the individual microparticles following milling has a volume average size reduction of at least 15% and a number average size reduction of no more than 75%. In one embodiment, particles are fed to the jet mill via a feeders and a suitable gas, preferably dry nitrogen, is used to feed and grind the microparticles through the mill. Grinding and feed gas pressures can be adjusted based on the material characteristics. Microparticle throughput depends on the size and capacity of the mill. The milled microparticles can be collected by filtration or, more preferably, cyclone.

Processing into Oral Dosage Form

The milled dry powder blend is converted to at least one oral dosage form known in the art. The skilled artisan can envision many ways of processing the particle blends in the methods and for the formulations described herein, and the following examples describing how oral dosage forms may be produced are not intended to limit in any way the methods and formulations described and claimed herein. In various embodiments, the milled blend of particles is processed into a powder- or pellet-filled capsule, a film, a conventional tablet, a modified or targeted delivery tablet, an orally disintegrating tablet or wafer, or a “sprinkle packet” (a packaged powder form suitable for application onto food or into beverage immediately before consumption by the patient; each packet typically is a unit dose). In another embodiment, the milled pharmaceutical formulation blend may be processed into a liquid suspension for oral administration.

As used herein, the term “orally disintegrating wafer” refers and includes orally disintegrating tablets (ODTs), wafers, films, or other solid preparations that rapidly disintegrate in the oral cavity, e.g., usually in a matter of a few seconds when placed on the tongue, when taken together with the saliva in the oral cavity or a small amount of water. In a preferred embodiment of the process, the milled blend is combined with suitable bulking agents, disintegrants, and other excipients to make the orally disintegrating wafer. Examples of these other excipients may include modified release polymers, waxes, coloring agents, sweeteners, flavoring agents, taste masking agents, or combinations thereof. In one embodiment, an oral disintegrating tablet pharmaceutical formulation is provided that includes a mixture of granules formed by granulation of a milled blend of (i) microparticles which comprise a pharmaceutical agent, and (ii) excipient particles; particles of at least one sugar; and particles of at least one disintegrant, wherein the mixture has been compressed into a tablet or wafer form.

In one embodiment, the milled blend is processed into tablets using standard tabletting methods. Tablets are a solid pharmaceutical dosage form containing the pharmaceutical agent, with or without suitable excipients and prepared by compression or molding methods. Compressed tablets are prepared using a tablet press from powders or granules in combination with excipients such as diluents, binders, disintegrants, lubricants, and glidants. Other excipients, such as modified release polymers, waxes, coloring agents, sweeteners, flavoring agents, or combinations thereof, can also be added.

Tablets or capsules can be further coated with polymer or sugar films or enteric or sustained release polymer coatings. Layered tablets can be prepared by compressing additional powders or granules on a previously prepared tablet for immediate or modified release.

The dry powder milled blends can be processed into granules using wet granulation methods, dry granulation methods, melt extrusion or spray drying of the powder dispersed into an appropriate liquid. The granules can be filled into capsules, processed into tablets or further processed into pellets using spheronization equipment. Pellets can be directly filled into capsules or compressed into tablets.

In a preferred embodiment, a solid oral dosage form of a pharmaceutical agent is provided that includes a milled blend of microparticles of a pharmaceutical agent blended with particles of at least one excipient, which milled blend has been processed into a solid oral dosage form, wherein the size of the microparticles following reconstitution of the solid oral dosage form is not more than 300%, preferably not more than 200%, more preferably not more than 150%, of the size of the microparticles in the milled blend pre-processing.

The milled blend may optionally undergo additional processes before being finally made into an oral dosage form. Representative examples of such processes include lyophilization or vacuum drying to further remove residual solvents, temperature conditioning to anneal materials, size classification to recover or remove certain fractions of the particles (i.e., to optimize the size distribution), granulation, and spheronization.

The Particles and Formulation Components

The oral dosage formulations made as described herein include mixtures of particles. The mixture generally includes (1) microparticles or nanoparticles that comprise the pharmaceutical agent and that may optionally comprise a shell material, and (2) particles of at least one, and typically more than one, excipient material.

Particles

The particles comprising pharmaceutical agent that are provided as a starting material in the methods described herein can be provided in a variety of sizes and compositions. As used herein, the term “particles” includes microparticles and nanoparticles, as well as larger particles, e.g., up to 5 mm in the longest dimension. In a preferred embodiment, the particles are microparticles. As used herein, the term “microparticle” encompasses microspheres and microcapsules, as well as microparticles, unless otherwise specified, and denotes particles having a size of 1 to 1000 microns. As used herein, “nanoparticles” are particles having a size of 1 to 1000 nm. In various embodiments, the microparticles or nanoparticles of pharmaceutical agent in the milled pharmaceutical formulation blend have a volume average diameter of less than 100 μm, preferably less than 20 μm, more preferably less than 10 μm. For oral administration for delivery to the gastrointestinal tract, for dissolution on the tongue, and for buccal application, the particles forming the oral dosage form may have a number average diameter of between 0.5 μm and 5 mm. In one embodiment, the particles of the milled pharmaceutical formulation blend have a volume average diameter of between about 1 and 50 μm. In another embodiment, the particles of the milled pharmaceutical formulation blend have a volume average diameter of between 2 and 10 μm.

Microparticles may or may not be spherical in shape. Microparticles can be rod like, sphere like, acicular (slender, needle-like particle of similar width and thickness), columnar (long, thin particle with a width and thickness that are greater than those of an acicular particle), flake (thin, flat particle of similar length and width), plate (flat particle of similar length and width but with greater thickness than flakes), lath (long, thin, blade-like particle), equant (particles of similar length, width, and thickness, this includes both cubical and spherical particles), lamellar (stacked plates), or disc like. “Microcapsules” are defined as microparticles having an outer shell surrounding a core of another material, in this case, the pharmaceutical agent. The core can be gas, liquid, gel, solid, or a combination thereof. “Microspheres” can be solid spheres, can be porous and include a sponge-like or honeycomb structure formed by pores or voids in a matrix material or shell, or can include multiple discrete voids in a matrix material or shell.

In one embodiment, the particle is formed entirely of the pharmaceutical agent. In another embodiment, the particle has a core of pharmaceutical agent encapsulated in a shell. In yet another embodiment, the pharmaceutical agent is interspersed within a shell or matrix. In still another embodiment the pharmaceutical agent is uniformly mixed within the material comprising the shell or matrix.

The terms “size” or “diameter” in reference to particles refers to the number average particle size, unless otherwise specified. An example of an equation that can be used to describe the number average particle size (and is representative of the method used for the Coulter counter) is shown below:

$$\frac{{\sum }_{i = 1}^{p} {n}_{i} {d}_{i}}{{\sum }_{i = 1}^{p} {n}_{i}}$$

where n=number of particles of a given diameter (d).

As used herein, the term “volume average diameter” refers to the volume weighted diameter average. An example of an equation that can be used to describe the volume average diameter, which is representative of the method used for the Coulter counter is shown below:

$${[ \frac{{\sum }_{i = 1}^{p} {n}_{i} {d}_{i}^{3}}{{\sum }_{i = 1}^{p} {n}_{i}} ]}^{1 / 3}$$

where n=number of particles of a given diameter (d).

Another example of an equation that can be used to describe the volume mean, which is representative of the equation used for laser diffraction particle analysis methods, is shown below:

$$\frac{\sum {d}^{4}}{\sum {d}^{3}}$$

where d represents diameter.

When a Coulter counter method is used, the raw data is directly converted into a number based distribution, which can be mathematically transformed into a volume distribution. When a laser diffraction method is used, the raw data is directly converted into a volume distribution, which can be mathematically transformed into a number distribution.

In the case of a non-spherical particle, the particles can be analyzed using Coulter counter or laser diffraction methods, with the raw data being converted to a particle size distribution by treating the data as if it came from spherical particles. If microscopy methods are used to assess the particle size for non-spherical particles, the longest axis can be used to represent the diameter (d), with the particle volume (Vₚ) calculated as:

$${V}_{p} = \frac{4 π {r}^{3}}{3}$$

where r is the particle radius (0.5 d), and a number mean and volume mean are calculated using the same equations used for a Coulter counter.

Particle size analysis can be performed on a Coulter counter, by light microscopy, scanning electron microscopy, transmission electron microscopy, laser diffraction methods, light scattering methods or time of flight methods. Where a Coulter counter method is described, the powder is dispersed in an electrolyte, and the resulting suspension analyzed using a Coulter Multisizer II fitted with a 50-μm aperture tube. Where a laser diffraction method is used, the powder is dispersed in an aqueous medium and analyzed using a Coulter LS230, with refractive index values appropriately chosen for the material being tested.

Analysis for agglomerates can be performed by visual evaluation of a suspension for the presence of macroscopic agglomerates, light microscopy for concentration of microscopic agglomerates, Coulter counter analysis or light scattering methods of analysis for particle size in suspension. A decrease in the particle size in suspension based on a volume mean indicates a decreased level of agglomerates.

1. Pharmaceutical Agent

The pharmaceutical agent is a therapeutic, diagnostic, or prophylactic agent. It may be an active pharmaceutical ingredient (API), and may be referred to herein generally as a “drug” or “active agent.” The pharmaceutical agent may be present in an amorphous state, a crystalline state, or a mixture thereof. The pharmaceutical agent may be labeled with a detectable label such as a fluorescent label, radioactive label or an enzymatic or chromatographically detectable agent.

The methods described herein advantageously can be used with pharmaceutical agents having low aqueous solubility, for example, where the pharmaceutical agent has a solubility in water of less than 10 mg/mL at 25° C.

The methods can be applied to a wide variety of therapeutic, diagnostic and prophylactic agents that may be suitable for oral administration. Representative examples of suitable drugs include the following categories and examples of drugs and alternative forms of these drugs such as alternative salt forms, free acid forms, free base forms, and hydrates:

analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, and meprobamate);

antiasthmatics;

antibiotics (e.g., neomycin, streptomycin, chloramphenicol, cephalosporin, ampicillin, penicillin, tetracycline, and ciprofloxacin);

antidepressants (e.g., nefopam, oxypertine, doxepin, amoxapine, trazodone, amitriptyline, maprotiline, phenelzine, desipramine, nortriptyline, tranylcypromine, fluoxetine, imipramine, imipramine pamoate, isocarboxazid, trimipramine, and protriptyline);

antidiabetics (e.g., biguanides and sulfonylurea derivatives);

antifungal agents (e.g., griseofulvin, ketoconazole, itraconizole, virconazole, amphotericin B, nystatin, and candicidin);

antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine);

anti-inflammatories (e.g., (non-steroidal) celecoxib, rofecoxib, indomethacin, ketoprofen, flurbiprofen, naproxen, ibuprofen, ramifenazone, piroxicam, (steroidal) cortisone, dexamethasone, fluazacort, hydrocortisone, prednisolone, and prednisone);

antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan);

antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene);

immunosuppressive agents (e.g., cyclosporine, azathioprine, mizoribine, and FK506 (tacrolimus), sirolimus);

antimigraine agents (e.g., ergotamine, propanolol, and dichloralphenazone);

sedatives/hypnotics (e.g., barbiturates such as pentobarbital, pentobarbital, and secobarbital; and benzodiazapines such as flurazepam hydrochloride, and triazolam);

antianginal agents (e.g., beta-adrenergic blockers; calcium channel blockers such as nifedipine, and diltiazem; and nitrates such as nitroglycerin, and erythrityl tetranitrate);

antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, lithium citrate, prochlorperazine, aripiprazole, and risperdione);

antimaniac agents (e.g., lithium carbonate);

antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, flecainide acetate, tocainide, and lidocaine);

antiarthritic agents (e.g., phenylbutazone, sulindac, penicillamine, salsalate, piroxicam, azathioprine, indomethacin, meclofenamate, gold sodium thiomalate, ketoprofen, auranofin, aurothioglucose, and tolmetin sodium);

antigout agents (e.g., colchicine, and allopurinol);

anticoagulants (e.g., heparin, low molecular weight heparin, desirudin, heparin sodium, and warfarin sodium);

thrombolytic agents (e.g., urokinase, streptokinase, and alteplase);

antifibrinolytic agents (e.g., aminocaproic acid);

hemorheologic agents (e.g., pentoxifylline);

antiplatelet agents (e.g., aspirin, clopidogrel);

anticonvulsants (e.g., valproic acid, divalproex sodium, phenyloin, phenyloin sodium, clonazepam, primidone, phenobarbitol, carbamazepine, amobarbital sodium, methsuximide, metharbital, mephobarbital, paramethadione, ethotoin, phenacemide, secobarbitol sodium, clorazepate dipotassium, oxcarbazepine and trimethadione);

antiparkinson agents (e.g., ethosuximide);

antihistamines/antipruritics (e.g., hydroxyzine, diphenhydramine, chlorpheniramine, brompheniramine maleate, cyproheptadine hydrochloride, terfenadine, clemastine fumarate, azatadine, tripelennamine, dexchlorpheniramine maleate, methdilazine);

agents useful for calcium regulation (e.g., calcitonin, and parathyroid hormone);

antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, clarithromycin and colistin sulfate);

antiviral agents (e.g., interferons, zidovudine, amantadine hydrochloride, ribavirin, and acyclovir);

antimicrobials (e.g., cephalosporins such as ceftazidime; penicillins; erythromycins; and tetracyclines such as tetracycline hydrochloride, doxycycline hyclate, and minocycline hydrochloride, azithromycin, clarithromycin);

anti-infectives (e.g., GM-CSF);

bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; salbutamol; ipratropium bromide; ketotifen; salmeterol; xinafoate; terbutaline sulfate; theophylline; nedocromil sodium; metaproterenol sulfate; albuterol);

corticosteroids (e.g., beclomethasone dipropionate (BDP), beclomethasone dipropionate monohydrate; budesonide, triamcinolone; flunisolide; fluticasone proprionate; mometasone);

steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium);

hypoglycemic agents (e.g., human insulin, purified beef insulin, purified pork insulin, glyburide, chlorpropamide, glipizide, tolbutamide, and tolazamide);

hypolipidemic agents (e.g., clofibrate, dextrothyroxine sodium, probucol, pravastitin, atorvastatin, lovastatin, and niacin);

proteins (e.g., DNase, alginase, superoxide dismutase, and lipase);

nucleic acids (e.g., sense or anti-sense nucleic acids encoding any therapeutically useful protein, including any of the proteins described herein); as useful for erythropoiesis stimulation (e.g., erythropoietin); antiulcer/antireflux agents (e.g., famotidine, cimetidine, and ranitidine hydrochloride); antinauseants/antiemetics (e.g., meclizine hydrochloride, nabilone, prochlorperazine, dimenhydrinate, promethazine hydrochloride, thiethylperazine, and scopolamine); oil-soluble vitamins (e.g., vitamins A, D, F, K, and the like); as well as other drugs such as mitotane, halonitrosoureas, anthrocyclines, and ellipticine. A description of these and other classes of useful drugs and a listing of species within each class can be found in Martindale, The Extra Pharmacopoeia, 30th Ed. (The Pharmaceutical Press, London 1993).

Examples of drugs useful in the methods and formulations described herein include ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleuklin, erythropoietin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotideo acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, calcitonin, and ipratropium bromide. These drugs are generally considered water-soluble.

Other examples of possible drugs include albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, n icardipine hydrochloride, zolpidem tartrate, amlodipine besylate, ethinyl estradiol, omeprazole, rubitecan, amlodipine besylate/benazepril hydrochloride, etodolac, paroxetine hydrochloride, paclitaxel, atovaquone, felodipine, podofilox, paricalcitol, betamethasone dipropionate, fentanyl, pramipexole dihydrochloride, Vitamin D₃ and related analogues, finasteride, quetiapine fumarate, alprostadil, candesartan, cilexetil, fluconazole, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, etodolac, triamcinolone acetate, estradiol, ursodiol, nelfinavir mesylate, indinavir, beclomethasone dipropionate, oxaprozin, flutamide, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam.

In one embodiment, the pharmaceutical agent used in the methods and formulations described herein is a hydrophobic compound, particularly a hydrophobic therapeutic agent. Examples of such hydrophobic drugs include celecoxib, rofecoxib, pactitaxel, docetaxel, acyclovir, alprazolam, amiodaron, amoxicillin, anagrelide, bactrim, biaxin, budesonide, bulsulfan, carbamazepine, ceftazidime, cefprozil, ciprofloxicin, clarithromycin, clozapine, cyclosporine, diazepam, estradiol, etodolac, famciclovir, fenofibrate, fexofenadine, gemcitabine, ganciclovir, itraconazole, lamotrigine, loratidine, lorazepam, meloxicam, mesalamine, minocycline, modafinil, nabumetone, nelfinavir mesylate, olanzapine, oxcarbazepine, phenyloin, propofol, ritinavir, SN-38, sulfamethoxazol, sulfasalazine, tracrolimus, tiagabine, tizanidine, trimethoprim, valium, valsartan, voriconazole, zafirlukast, zileuton, and ziprasidone.

In another embodiment, the pharmaceutical agent used in the methods and formulations described herein is a contrast agent for diagnostic imaging. For example, the diagnostic agent may be an imaging agent useful in positron emission tomography (PET), computer assisted tomography (CAT), single photon emission computerized tomography, x-ray, fluoroscopy, magnetic resonance imaging (MRI), or ultrasound imaging. Microparticles loaded with these agents can be detected using standard techniques available in the art and commercially available equipment. Examples of suitable materials for use as MRI contrast agents include soluble iron compounds (ferrous gluconate, ferric ammonium citrate) and gadolinium-diethylenetriaminepentaacetate (Gd-DTPA). In another example, the diagnostic agent containing particles comprise barium for oral administration.

2. Shell Material

The particles that include the pharmaceutical agent may also include a shell material. The shell material can be water soluble or water insoluble, degradable or non-degradable, erodible or non-erodible, natural or synthetic, depending for example on the particular oral dosage form selected and release kinetics desired. Representative examples of types of shell materials include polymers, amino acids, sugars, proteins, carbohydrates, and lipids. Polymeric shell materials can be degradable or non-degradable, erodible or non-erodible, natural or synthetic. Non-erodible polymers may be used for oral administration. In general, synthetic polymers may be preferred due to more reproducible synthesis and degradation. Natural polymers also may be used. A polymer may selected based on a variety of performance factors, including shelf life, the time required for stable distribution to the site (e.g., in the gastrointestinal tract) where delivery is desired, degradation rate, mechanical properties, and glass transition temperature of the polymer.

Representative examples of synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxyethyl cellulose, cellulose triacetate, and cellulose sulphate sodium salt jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof. Examples of preferred non-biodegradable polymers include ethylene vinyl acetate, poly(meth)acrylic acid, polyamides, copolymers and mixtures thereof.

Examples of preferred natural polymers include proteins such as albumin and prolamines, for example, zein, and polysaccharides such as alginate, cellulose and polyhydroxyalkanoates, for example, polyhydroxybutyrate. The in vivo stability of the matrix can be adjusted during the production by using polymers such as polylactide-co-glycolide copolymerized with polyethylene glycol (PEG). PEG, if exposed on the external surface, may extend the time these materials circulate post intravascular administration, as it is hydrophilic and has been demonstrated to mask RES (reticuloendothelial system) recognition.

Bioadhesive polymers can be of particular interest for use in targeting of mucosal surfaces (e.g., in the gastrointestinal tract, mouth). Examples of these include polyanhydrides, polyacrylic acid, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Representative amino acids that can be used in the shell include both naturally occurring and non-naturally occurring amino acids. The amino acids can be hydrophobic or hydrophilic and may be D amino acids, L amino acids or racemic mixtures. Amino acids that can be used include glycine, arginine, histidine, threonine, asparagine, aspartic acid, serine, glutamate, proline, cysteine, methionine, valine, leucine, isoleucine, tryptophan, phenylalanine, tyrosine, lysine, alanine, and glutamine. The amino acid can be used as a bulking agent, or as an anti-crystallization agent for drugs in the amorphous state, or as a crystal growth inhibitor for drugs in the crystalline state or as a wetting agent. Hydrophobic amino acids such as leucine, isoleucine, alanine, glycine, valine, proline, cysteine, methionine, phenylalanine, tryptophan are more likely to be effective as anticrystallization agents or crystal growth inhibitors. In addition, amino acids can serve to make the shell have a pH dependency that can be used to influence the pharmaceutical properties of the shell such as solubility, rate of dissolution or wetting.

The shell material can be the same or different from the excipient material.

Excipients, Bulking Agents

The drug particles are blended with one or more excipients particles. The term “excipient” refers to any non-active ingredient of the formulation intended to facilitate handling, stability, wettability, release kinetics, and/or oral administration of the pharmaceutical agent. The excipient may be a pharmaceutically acceptable carrier or a hulking agent as known in the art. The excipient may comprise a shell material, protein, amino acid, sugar or other carbohydrate, starch, lipid, or combination thereof. In one embodiment, the excipient is in the form of microparticles. In one embodiment, the excipient microparticles may have a volume average size between about 5 and 500 μm.

In one embodiment, the excipient in the methods and formulations described herein is a pre-processed excipient. A pre-processed excipient is one that initially cannot be readily handled in a dry powder form that is converted into a form suitable for dry powder processing. A preferred pre-processing process is described above. In preferred embodiments, at least one excipient of the pre-processed excipient comprises a liquid, waxy, non-crystalline compound, or other non-friable compound. In a preferred embodiment, the non-friable excipient comprises a surfactant, such as a waxy or liquid surfactant. By “liquid,” it is meant that the material is a liquid at ambient temperature and pressure conditions (e.g., 15-25° C. and atmospheric pressure) Examples of such surfactants include docusate sodium (DSS) and polysorbates (Tweens). In a preferred embodiment, the surfactant is a Tween or other hydrophilic surfactant. The pre-processed excipient further includes at least one bulking agent. In preferred embodiments, the bulking agent comprises at least one sugar, sugar alcohol, starch, amino acid, or combination thereof. Examples of suitable bulking agents include lactose, sucrose, maltose, mannitol, sorbitol, trehalose, galactose, xylitol, erythritol, and combinations thereof.

In one particular embodiment of the methods described herein, mannitol and TWEEN™ 80 are blended in the presence of water and the water is then removed by spray-drying or lyophilization, yielding a pre-processed excipient of mannitol and TWEEN™ 80. The pre-processed mannitol TWEEN™ 80 blend is then blended with microparticles formed of or including an API.

In another particular embodiment, mannitol and DSS are blended in the presence of water, and the water is then removed by spray-drying or lyophilization, yielding a pre-processed excipient of mannitol and DSS. The pre-processed mannitol/DSS blend is then blended with microparticles formed of or including an API.

Representative amino acids that can be used as excipients include both naturally occurring and non-naturally occurring amino acids. The amino acids can be hydrophobic or hydrophilic and may be D amino acids, L amino acids or racemic mixtures. Amino acids which can be used include glycine, arginine, histidine, threonine, asparagine, aspartic acid, serine, glutamate, proline, cysteine, methionine, valine, leucine, isoleucine, tryptophan, phenylaianine, tyrosine, lysine, alanine, and glutamine. The amino acid can be used as a bulking agent, as a wetting agent, or as a crystal growth inhibitor for drugs in the crystalline state. Hydrophobic amino acids such as leucine, isoleucine, alanine, glycine, valine, proline, cysteine, methionine, phenylalanine, tryptophan are more likely to be effective as crystal growth inhibitors. In addition, amino acids can serve to make the matrix have a pH dependency that can be used to influence the pharmaceutical properties of the matrix, such as solubility, rate of dissolution, or wetting.

Examples of excipients include surface active agents, dispersants, osmotic agents, binders, disintegrants, glidants, diluents, color agents, flavoring agents, sweeteners, and lubricants. Examples include sodium desoxycholate; sodium dodecylsulfate; polyoxyethylene sorbitan fatty acid esters, e.g., polyoxyethylene 20 sorbitan monolaurate (TWEEN™ 20), polyoxyethylene 4 sorbitan monolaurate (TWEEN™ 21), polyoxyethylene 20 sorbitan monopalmitate (TWEEN™ 40), polyoxyethylene 20 sorbitan monooleate (TWEEN™ 80); polyoxyethylene alkyl ethers, e.g., polyoxyethylene 4 lauryl ether (BRIJ™ 30), polyoxyethylene 23 lauryl ether (BRIJ™ 35), polyoxyethylene 10 oleyl ether (BRIJ™ 97); polyoxyethylene glycol esters, e.g., poloxyethylene 8 stearate (MYRj™ 45), poloxyethylene 40 stearate (MYRJ™ 52); Tyloxapol; Spans; and mixtures thereof. Examples of binders include starch, gelatin, sugars, gums, polyethylene glycol, ethylcellulose, waxes and polyvinylpyrrolidone. Examples of disintegrants (including super disintegrants) includes starch, clay, celluloses, croscarmelose, crospovidone and sodium starch glycolate. Examples of glidants include colloidal silicon dioxide and talc. Examples of diluents include dicalcium phosphate, calcium sulfate, lactose, cellulose, kaolin, mannitol, sodium chloride, dry starch and powdered sugar. Examples of lubricants include talc, magnesium stearate, calcium stearate, stearic acid, hydrogenated vegetable oils, and polyethylene glycol.

The invention can further be understood with reference to the following non-limiting examples.

### EXAMPLES

The following materials were used in the examples: mannitol (Spectrum Chemicals, New Brunswick, N.J., unless otherwise indicated), TWEEN™ 80 (Spectrum Chemicals, New Brunswick, N.J.), DSS (Docusate Sodium, Cytec Industries, West Paterson, N.J.), fenofibrate (Onbio, Ontario, Canada), celecoxib (Onbio, Ontario, Canada), SDS (Sodium Dodecyl Sulfate, Spectrum Chemicals, New Brunswick, N.J.), Plasdone S630 (ISP Technologies Inc., Wayne, N.J.), Hypromellose (HPMC, Pharmacoat 606, Sin-Etsu Chemical Co. Ltd., Tokyo, Japan), Xylitol (Xylisorb 700, Roquette America Inc., Keokuk, Iowa), and Crospovidone (Polyplasdone XL, ISP Technologies Inc., Wayne, N.J. The TWEENT™ 80 is hereinafter referred to as “Tween80.”

A TURBULA™ inversion mixer (model: T2F) was used for blending. A Hosokawa Alpine Aeroplex Spiral Jet Mill (model: 50AS) or a Fluid Energy Aljet Jet Mill were used for milling, with dry nitrogen gas as the injector and grinding gases. In the studies, the dry powder was fed manually into the jet mill, and hence the powder feed rate was not constant. Although the powder feeding was manual, the feed rate was calculated to be approximately 1 to 5 g/min for all of the studies. Feed rate is the ratio of total material processed in one batch to the total batch time. Particle size measurement of the jet milled samples, unless otherwise indicated, was conducted using a Coulter Multisizer II with a 50 μm aperture.

### Example 1

### Jet Milling a Blend of PLGA Microparticles with Pre-Processed Excipient Particles Comprising Tween80 and Mannitol

Blending was conducted in two steps: a first step in which an excipient was pre-processed into a dry powder form and a second step in which the particles (representing particles of a pharmaceutical agent) were combined with the particles of pre-processed excipient. In the first step, mannitol and Tween80 were blended in liquid form, wherein 500 mL of Tween80/mannitol vehicle was prepared from Tween80, mannitol, and water. The vehicle was frozen and then subjected to vacuum drying, yielding a powder comprised of Tween80 homogeneously dispersed with the mannitol. In the second step, poly(lactide-co-glycolide) (50:50) (“PLGA”) microparticles (which represented the pharmaceutical agent particles) were combined with the mannitol/Tween80 blend and mixed in a tumbler mixer to yield a dry blended powder. The PLGA microparticles had an Xn=2.83 micron and Xv 8.07 micron. The dry blended powder was then fed manually into the Hosokawa jet mill, operated at three different sets of operating conditions. The resulting milled blend samples were analyzed for particle size. For comparison, a control sample (blended but not jet milled) was similarly analyzed. The Coulter Multisizer II results are shown in Table 1.

TABLE 1
Results of Particle Size Analysis

| | Number Avg. | Volume Avg. |
|---|---|---|
| Sample | Particle Size, Xₙ (μm) | Particle Size, X_{y} (μm) |
| Control | 2.78 | 8.60 |
| 2.1 | 1.98 | 4.52 |
| 2.3 | 1.99 | 4.11 |
| 2.3 | 1.93 | 3.37 |

The results demonstrate the advantage to dispersibility (as assessed by volume mean (Xv), with a smaller Xv being an indicator of decreased agglomerates) offered by milled blend formulations.

### Example 2

### Jet Milling of Celecoxib/Excipient Blend for Improved Microparticle Dispersibility

Mannitol (89.3 g, Pearlitol 100SD from Roquette America Inc., Keokuk, Iowa), sodium lauryl sulfate (3.46 g), celecoxib (149.0 g), and hypromellose-606 (9.35 g) were added to a stainless steel jar. The jar was then set in a TURBULA™ mixer for 90 minutes at 96 min⁻¹, yielding a dry blended powder. The dry blended powder then was fed manually into a Fluid Energy Aljet jet mill (injector gas pressure 8.0 bar, grinding gas pressure 4.0 bar) to produce well dispersing microparticles.

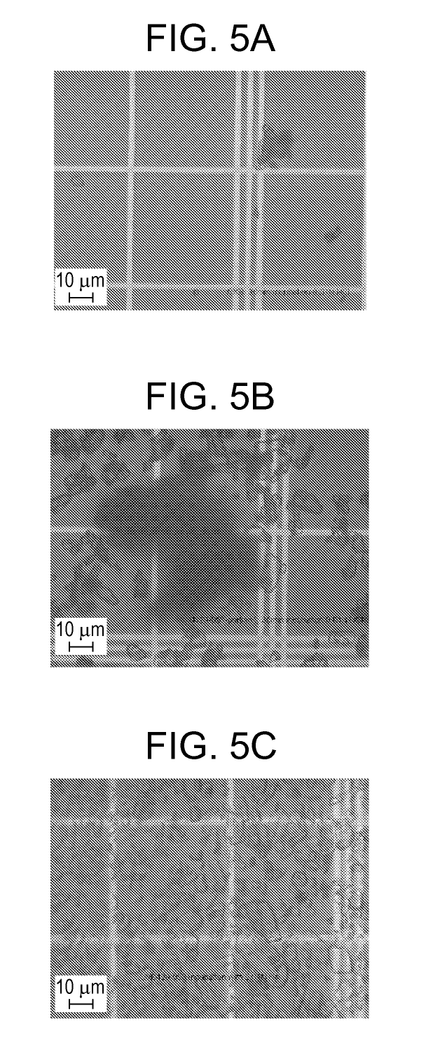

The unprocessed celecoxib, the blended celecoxib, and the jet milled blended celecoxib were analyzed using visual inspection and by light microscopy (performed on a hemacytometer slide) following reconstitution in 0.01N HCl. FIGS. 5A, 5B, and 5C show the particles of the bulk celecoxib, the blended powder, and the jet-milled blended powder, respectively. The quality of the suspensions are described in Table 2.

TABLE 2
Results of Visual Evaluation of Dispersibility

| Sample | Visual Evaluation of Suspension |
|---|---|
| Celecoxib/no blending or jet milling | Poor suspension containing many |
| | unwetted macroscopic articles |
| Blended celecoxib/no jet milling | Mixture of a fine suspension and |
| | many macroscopic particles |
| Blended & jet milled celecoxib | A fine suspension containing a few |
| | small macroscopic articles |

Jet milling of blended celecoxib particles led to a powder which was better dispersed, as indicated by the resulting fine suspension with a few macroscopic particles. This suspension was better than the suspensions of the unprocessed celecoxib powder and the blended celecoxib powder. The light microscope images of the suspensions indicate no significant change to individual particle morphology, just to the ability of the individual particles to disperse as indicated by the more uniform size and increased number of suspended particles following both blending and jet milling as compared to the two other particle samples.

### Example 3

### Granulation and Tabletting of a Milled Blend Comprising Fenofibrate and a Pre-processed Excipient

To create a pre-processed excipient, a solution of mannitol (267.7 g, Pearlitol 100SD) and DSS (32.16 g) in 2264 g of water was prepared. The solution was frozen and lyophilized, and the resulting powder was screened through an 850 μm sieve prior to blending with the fenofibrate particles.

A dry powder blend formulation was prepared by one of three different processes. The blend included fenofibrate, mannitol, DSS, and Plasdone S630 in a 10:10:1.2:2.0 ratio, where the mannitol and DSS were in the form of the pre-processed excipient described above. The total blend amount was 150 g. The three processes were (1: API Blend) blending the fenofibrate and excipient particles without milling, (2: Blend of JM API) separately milling the fenofibrate particles and then blending the milled particles with excipient particles, or (3: JM API Blend) blending the fenofibrate and excipient particles and then milling the resulting blend. For blending, the materials were added to a stainless steel jar. The jar was then set in a TURBULA™ mixer for 30 minutes at 96 min⁻¹, yielding a dry blended powder. For jet milling, the material was fed manually into a Fluid Energy Aljet jet mill (injector gas pressure 8.0 bar, grinding gas pressure 4.0 bar).

The resulting materials were reconstituted in 0.01N HCl, and analyzed for particle size using a Coulter LS230 Laser Diffraction Particle Size Analyzer. The particles sizes were compared for the three processes, and the results are shown below in Table 3.

The JM API Blend was granulated using a Vector MFL.01 fluid bed processor. DI water was top sprayed over fluidizing bed of jet milled blend powder from above to form granules. The following process conditions were used: the liquid feed rate ranged from 1 g/min to 2 g/min, the fluid bed process gas was supplied at a rate in the range of 80 LPM to 130 LPM, the nozzle atomization pressure rate of 10.1 psi, the inlet temperature in the range of 50° C. to 65° C., and the outlet temperature in the range of 20° C. to 35° C.

The powders (approximately 530 mg) were then compacted using the automatic Carver Tablet Press (14 mm standard concave tooling, approximately 1000-1100 lbs pressure) to produce compacts for particle size analysis using the Coulter LS230.

The powders (2.1 g) were also blended with xylitol (2.1 g) and crospovidone (0.7 g) in a steel jar. The jar was then set in a TURBULA™ mixer for 10 minutes at 96 min⁻¹, yielding a dry blended powder. The resultant blends from above (approximately 1082 mg per tablet) were then tabletted using the automatic Carver Tablet Press (14 mm standard concave tooling, approximately 900-1300 lbs pressure) to produce orally disintegrating tablets. The tablets were analyzed for disintegration using a Electrolab-Disintegration Tester from GlobePharma (in 800 mL deionized water at 37° C.).

Table 3 below shows the particle size data from light scattering analysis using a Coulter LS230 (where “Xv” is volume mean, “%<90” is the size at which 90% of the volume is less than that size, and “σ” is standard deviation) for the blends, granules, compacts and the disintegration time of the orally disintegrating tablets.

TABLE 3
Results of Particle Size Analysis for Granulation and Tabletting

| | Pre-compaction | | Post-compaction | | Disintegration | |
|---|---|---|---|---|---|---|
| | (μm) | | (μm) | | Time (s) | |
| Sample | Xv | % < 90 | Xv | % < 90 | Mean | σ |
| Blend of API and Preprocessed excipient | 118.05 | 182.18 | 110.655 | 192.65 | 32.0 | 5.2 |
| Blend of JM API and Pre-processed | 22.09 | 59.69 | 21.905 | 56.435 | 43.33 | 5.77 |
| excipient | | | | | | |
| JM API blend (Jet Milled Blend of API | 5.618 | 12.075 | 8.068 | 16.38 | 120.0 | 20.0 |
| and Pre-processed excipient) | | | | | | |
| Granulated JM API blend (Jet Milled | 6.773 | 13.845 | 11.725 | 27.945 | 31.67 | 2.89 |
| Blend of API and Pre-processed excipient) | | | | | | |

The results indicate that the processing method impacts the suspension quality. The results demonstrate the advantage to dispersibility (as assessed by volume mean (Xv), with a smaller Xv being an indicator of decreased agglomerates) offered by milled blend formulations as compared to formulations to the formulations made by the other methods. The results also demonstrate that rapidly disintegrating tablets can be formed from granules of a JM API blend.

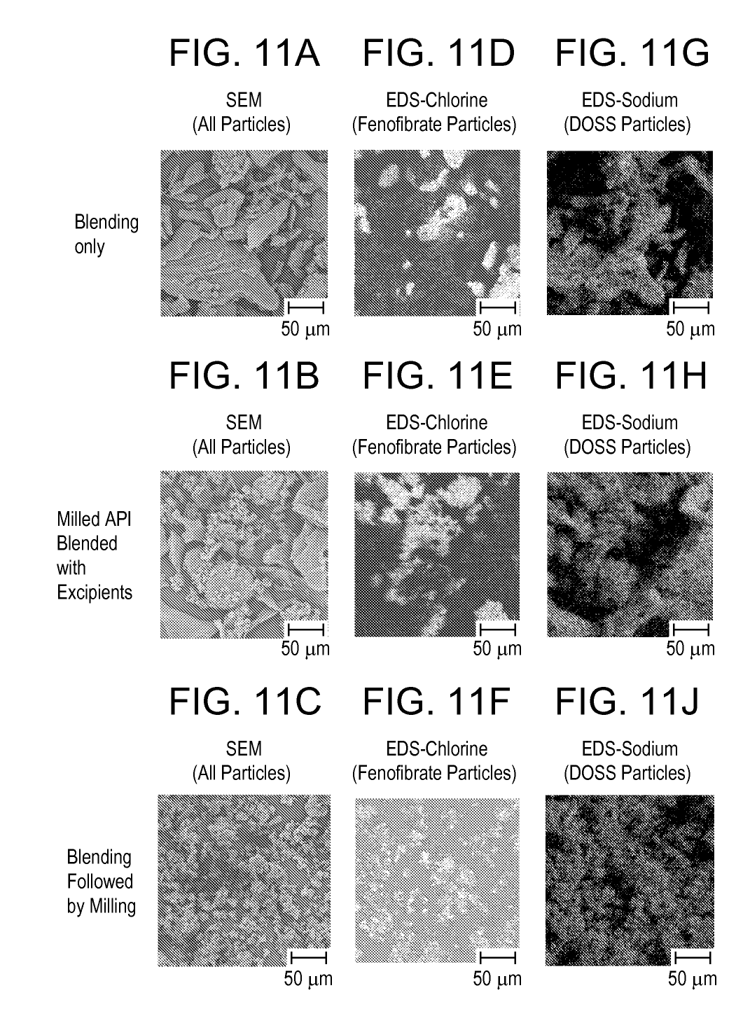

FIGS. 11A, 11B, and 11C are Scanning Electron Microscopy (SEM) images of the differently processed bulk powders. FIGS. 11D, 11E, and 11F are Energy Dispersive X-Ray Spectroscopy (EDS) images with analysis for chlorine (only present in fenofibrate) of the differently processed bulk powders. FIGS. 11G, 11H, and 11J are EDS images with analysis for sodium (only present in DSS) of the differently processed bulk powders. The images illustrate that the processes used and the order of processing affected the uniformity of the distribution of the fenofibrate particles among the excipient particles in the dry powder state. FIGS. 11A, 11D, and 11G shows the API/excipient blend (made without jet-milling) in which the native, untreated API particles (in a broad particle size range) were unevenly distributed in the powder mixture. When jet milling of fenofibrate was performed prior to blending with excipients, fenofibrate-rich areas (seen as clusters of smaller chlorine containing particles) and excipient rich areas (larger particles) were observed, as shown in FIGS. 11B, 11E, and 11H. When blending was performed prior to jet milling, the fenofibrate was more uniformly distributed among the excipient particles, as shown in FIGS. 11C, 11F, and 11J.

### Example 4

### Comparison of Jet Milled Blend of Celecoxib with Non-Preprocessed or Pre-Processed Excipient Particles

Two blends were made containing celecoxib, mannitol (Pearlitol 100SD), Tween80 (Spectrum), and Plasdone-C15 in a 10:10:1:1 ratio. Sample 1 was made by jet milling a blend of celecoxib, mannitol, Tween80, and Plasdone-C15 directly (i.e., no pre-processing of excipients). Sample 2 was made by jet milling a blend of celecoxib and pre-processed mannitol/Tween80/Plasdone-C15. The mannitol and Tween80 were pre-processed, at a ratio of 10:1, by dissolution in water (85.2 g mannitol and 8.54 g Tween80 in 749 g water) followed by freezing and lyophilization. Each formulation was blended using a TURBULA™ mixer, to produce a dry blended powder. The resulting dry powder blend was then fed manually into a Fluid Energy Aljet jet mill, and observations were made of the ease of processing during milling. These observations are described in Table 4.

TABLE 4
Milling Observations Related to Ease of Processing

| Sample | Milling Comment |
|---|---|
| Jet milled blend of celecoxib and | The mill clogged many times. Near |
| non-preprocessed excipients | the gasket of the jet mill, many |
| | aggregates (like granules) were |
| | observed. |
| Jet milled blend of celecoxib and pre- | The mill clogged a few times. |
| processed excipients | |

The material made with pre-processed excipient was easier to mill than the material made with the non-preprocessed excipient.

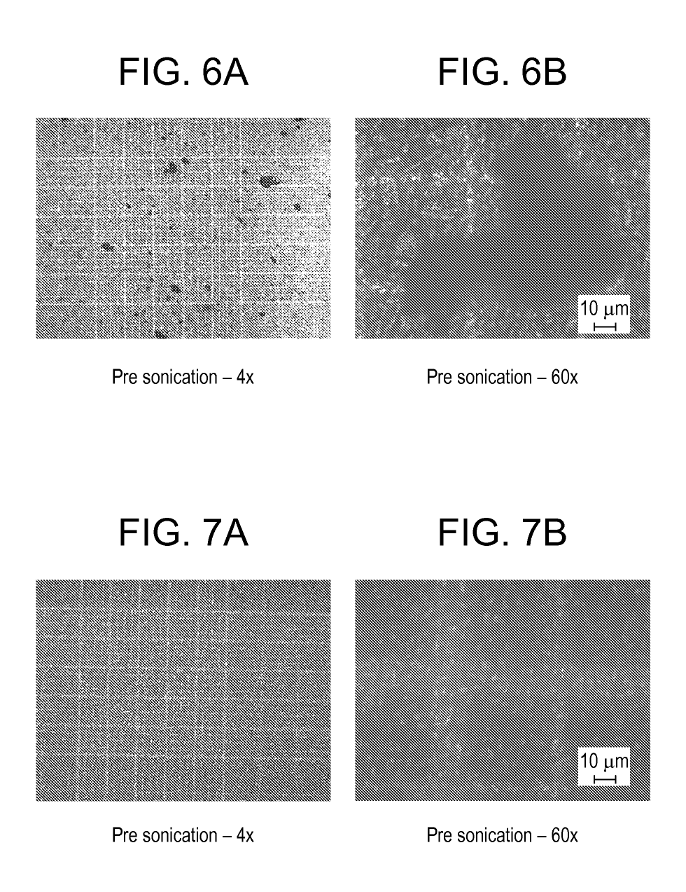

The resulting milled blends of Sample 1 and 2 were reconstituted with water and examined by microscopy. Agglomerates were observed in the formulation containing non-lyophilized mannitol/Tween80. However, large agglomerates were not visible for the material that contained lyophilized mannitol/Tween80/PVP, indicating that pre-processing of the Tween80 excipient resulted in improved dispersal, as shown in FIGS. 6A-B (Sample 1) and FIG. 7A-B (Sample 2).

### Example 5

### Microparticle Dispersibility Comparison of Reconstituted Celecoxib Blend Formulations with Pre-processed Mannitol, Plasdone-C15, and Tween80

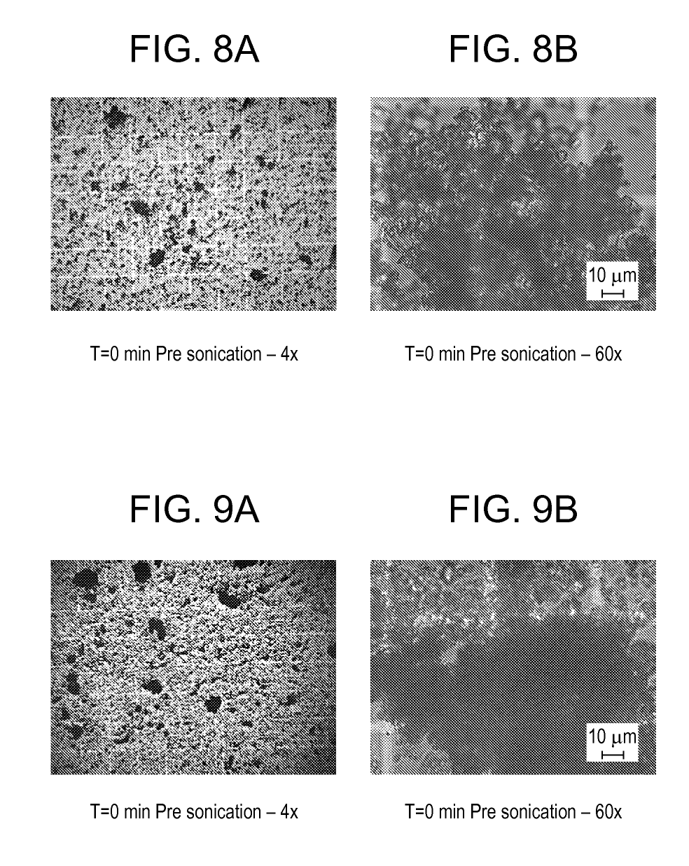

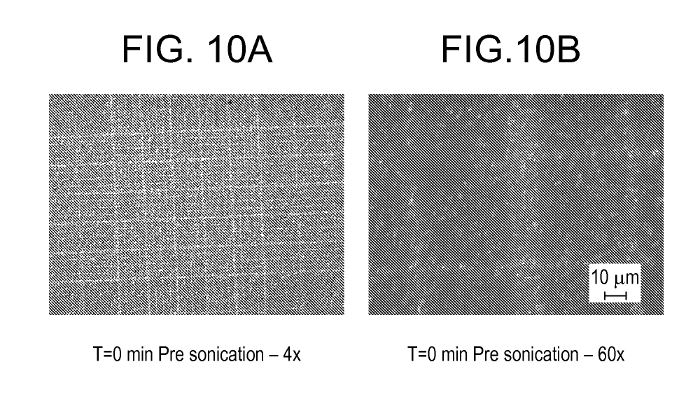

A dry powder blend formulation was prepared by one of three different processes and then reconstituted in water. The dry powder blend consisted of celecoxib, mannitol (Pearlitol 100SD), Plasdone-C15, and Tween80 at a ratio of 5:10:1:1. The mannitol and the Tween80 were pre-processed, at a ratio of 10:1, by dissolution in water (18 g mannitol and 1.8 g Tween80 in 104 mL water) followed by freezing at −80° C. and lyophilization, yielding pre-processed excipient particles. The three processes compared were (1) blending the celecoxib and pre-processed excipient particles without milling, (2) separately milling the celecoxib particles and then blending the milled particles with pre-processed excipients, or (3) blending the celecoxib and pre-processed excipient particles and then milling the resulting blend. The resulting blends were reconstituted in water using shaking, and analyzed by light scattering using an LS230 (Beckman Coulter, Fullerton, Calif.). The particles' sizes from each of the three processes were compared. The size results are shown in Table 5, along with visual evaluations of the quality of the suspensions. FIGS. 8A-B show the microscopy results of reconstituted celecoxib from a blend of excipient particles and celecoxib particles (Process 1). FIGS. 9A-B show the microscopy results of reconstituted celecoxib from a blend of excipient particles and milled celecoxib particles (Process 2). FIGS. 10A-B show the microscopy results of reconstituted celecoxib from a jet milled blend of excipient particles and celecoxib particles (Process 3).

TABLE 5
Results of Particle Size Analysis and Observations Following Reconstitution

| | Particle Size | | | |
|---|---|---|---|---|
| | Analysis | | | |
| | T = 0 | | | |
| | Post Reconstitution | | | |
| | Volume | % < 90 | Post Reconstitution | |
| Sample | mean (μm) | (μm) | T = 0 | T = 60 min |
| Celecoxib Particles | 56.27 | 156.95 | Fine suspension with many | Fine suspension with many |
| Blended | | | small macroparticles | small macroparticles |
| Blend of Jet Milled | 58.98 | 153.08 | Fine suspension with many | Fine suspension with many |
| Celecoxib Particles | | | small macroparticles | small macroparticles |
| Jet Milled Blend of | 5.45 | 9.12 | Fine suspension with very | Fine Suspension |
| Celecoxib Particles | | | few small macroparticles | |

These results strongly indicate that the processing method impacts the resulting suspension quality. The results also indicate the advantages offered by milled blend formulations as compared to the formulations made by the other methods.

Jet milling of blended celecoxib particles led to a powder which was better dispersed, as indicated by the resulting fine suspension with a few macroscopic particles. This suspension was better than the suspensions of the unprocessed celecoxib microparticles and the blended celecoxib microparticles.

The light microscope images (FIGS. 8-10) of the suspensions indicate no significant change to individual particle morphology, just to the ability of the individual particles to disperse as indicated by the more uniform size and increased number of suspended microparticles following both blending and jet milling as compared to the two other microparticle samples.

### Example 6

### Particle Size Comparison of Reconstituted Celecoxib Blend Formulations with Non-Preprocessed Mannitol, HPMC, and SDS

A dry powder blend formulation was prepared by one of three different processes. The blend included celecoxib, mannitol, HPMC, and SDS at a ratio of 10:6:0.63:0.35. The three processes were (1) blending the celecoxib and excipient particles without milling, (2) separately milling the celecoxib particles and then blending the milled particles with excipient particles, or (3) blending the celecoxib and excipient particles and then milling the resulting blend. The resulting blends were reconstituted in 0.01N HCl, and analyzed for particle size using a Coulter LS230. The particles sizes were compared for the three processes, and the results are shown below in Table 6.

TABLE 6
Results of Particle Size Analysis

| | Pre-sonication | | Post-sonication | |
|---|---|---|---|---|
| | Volume | % < 90 | Volume | % < 90 |
| Sample | mean (μm) | (μm) | mean (μm) | (μm) |
| Celecoxib Particles Blended | 12.63 | 20.86 | 11.03 | 19.2 |
| Blend of Jet Milled Celecoxib | 8.322 | 13.72 | 6.87 | 13.09 |
| Particles | | | | |
| Jet Milled Blend of Celecoxib | 5.15 | 9.26 | 5.17 | 9.32 |
| Particles | | | | |

The results again indicate that the processing method impacts the suspension quality. The results demonstrate the advantage offered by milled blend formulations as compared to the formulations made by the other methods.

### Example 7

### Granulation and Tabletting of a Milled Blend Comprising Celecoxib and a Non-Preprocessed Excipient

A dry powder blend formulation was prepared by one of three different processes. The blend included celecoxib, mannitol (Pearlitol 100SD), hypromellose-606, and sodium lauryl sulfate in a 10:6:0.63:0.35 ratio. The three processes were (1: API Blend) blending the celecoxib and excipient particles without milling, (2: Blend of JM API) separately milling the celecoxib particles and then blending the milled particles with excipient particles, or (3: JM API Blend) blending the celecoxib and excipient particles and then milling the resulting blend. For blending, the materials were added to a stainless steel jar. The total blend amount was 250 g for blending of the API and excipient particles, and 150 g for blending of the jet milled API with excipient particles. The jar was then set in a TURBULA™ mixer for 60 minutes at 96 min⁻¹, yielding a dry blended powder. For jet milling, the material was fed manually into a Fluid Energy Aljet jet mill (injector gas pressure 8.0 bar, grinding gas pressure 4.0 bar).

The JM API blend was granulated using a Vector MFL.01 fluid bed processor. DI water was top sprayed over fluidizing bed of jet milled blend powder from above to form granules. The following process conditions were used: the liquid feed rate ranged from 2.2 g/min to 3.2 g/min, the fluid bed process gas was supplied at a rate in the range of 80 LPM to 130 LPM, the nozzle atomization pressure was 10 psi, the inlet temperature was in the range of 55° C. to 70° C., and the outlet temperature was in the range of 19° C. to 25° C.

The powders (approximately 500 mg) were then compacted using the automatic Carver Tablet Press (14 mm standard concave tooling, approximately 1000-1100 lbs pressure) to produce compacts for particle size analysis using the Coulter LS230.

The powders (1.5 g) were also blended with xylitol (1 g) and crospovidone (0.5 g) in a steel jar. The jar was then set in a TURBULA™ mixer for 10 minutes at 96 min⁻¹, yielding a dry blended powder. The resultant blends from above (approximately 678 mg per tablet) were then tabletted using the automatic Carver Tablet Press (14 mm standard concave tooling, approximately 600-1200 lbs pressure) to produce orally disintegrating tablets. The tablets were analyzed for disintegration using a Electrolab-Disintegration Tester from GlobePharma (in 800 mL deionized water at 37° C.).

Table 7 below shows the particle size data (where “Xv” is volume mean, “%<90” is the size at which 90% of the volume is less than that size, and “σ” is standard deviation) for the granules, compacts and the disintegration time of the orally disintegrating tablets.

TABLE 7
Results of Particle Size Analysis for Granulation and Tabletting

| | Pre-compaction | | Post-compaction | | Disintegration | |
|---|---|---|---|---|---|---|
| | (μm) | | (μm) | | Time (s) | |
| Sample | Xv | % < 90 | Xv | % < 90 | Mean | σ |
| Blend of API and Non-Ike-processed | 12.63 | 20.86 | 12.79 | 32.97 | 33 | 3.06 |
| excipient | | | | | | |
| Blend of JM API ad Non-Pre-processed | 8.322 | 13.72 | 11.44 | 32.18 | 25 | 0 |
| excipient | | | | | | |
| JM API blend (Jet Milled Blend of API and | 5.15 | 9.26 | 11.31 | 26.22 | 42 | 28.73 |
| Non-Pre-processed excipient) | | | | | | |
| Granulated JM API blend (Jet Milled Blend | 5.67 | 10.20 | 6.11 | 13.76 | 32 | 4.16 |
| of API and Non-Pre-processed excipient) | | | | | | |

The results indicate that the processing method impacts the suspension quality. The results demonstrate the advantage to dispersibility (as assessed by volume mean (Xv), with a smaller Xv being an indicator of decreased agglomerates) offered by milled blend formulations as compared to formulations to the formulations made by the other methods. The results also demonstrate that rapidly disintegrating tablets can be formed from granules of a JM API blend.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

We claim:

1. A method for making an oral dosage form of a pharmaceutical agent, comprising the steps of:
a) providing particles which comprise a pharmaceutical agent;
b) blending the particles with particles of a pre-processed excipient to form a primary blend, wherein the pre-processed excipient is prepared by
i) dissolving a bulking agent and at least one non-friable excipient in a solvent to form an excipient solution, and
ii) removing the solvent from the excipient solution to form the pre-processed excipient in dry powder form;
c) milling the primary blend to form a milled pharmaceutical formulation blend, which comprises microparticles or nanoparticles of the pharmaceutical agent; and
d) processing the milled pharmaceutical formulation blend into a solid oral dosage form or liquid suspension for oral administration.

2. The method of claim 1, wherein the particles of step a) are microparticles.

3. The method of claim 1, wherein the milling comprises jet milling.

4. The method of claim 1, wherein the milled pharmaceutical formulation blend is processed into a solid oral dosage form selected from the group consisting of tablets, capsules, orally disintegrating wafers, and sprinkle packets.

5. The method of claim 1, wherein the bulking agent comprises at least one sugar, sugar alcohol, starch, amino acid, or combination thereof.

6. The method of claim 1, wherein the bulking agent is selected from the group consisting of lactose, sucrose, maltose, mannitol, sorbitol, trehalose, galactose, xylitol, erythritol, and combinations thereof.

7. The method of claim 1, wherein the non-friable excipient comprises a liquid, waxy, or non-crystalline compound.

8. The method of claim 1, wherein the non-friable excipient comprises a surfactant.

9. The method of claim 8, wherein the surfactant comprises a waxy or liquid surfactant.

10. The method of claim 8, wherein the surfactant comprises docusate sodium or a polysorbate.

11. The method of claim 1, wherein the step of removing the solvent comprises spray drying.

12. The method of claim 1, wherein the step of removing the solvent comprises lyophilization, vacuum drying, or freeze drying.

13. The method of claim 1, wherein the pre-processed excipient particles are milled before blending with the particles of step (a).

14. The method of claim 1, wherein the pharmaceutical agent has a solubility in water of less than 10 mg/mL at 25° C.

15. The method of claim 1, wherein the microparticles or nanoparticles of pharmaceutical agent in the milled pharmaceutical formulation blend have a volume average diameter of less than 100 μm.

16. The method of claim 1, wherein the microparticles or nanoparticles of pharmaceutical agent in the milled pharmaceutical formulation blend have a volume average diameter of less than 20 μm.

17. The method of claim 1, wherein the microparticles or nanoparticles of pharmaceutical agent in the milled pharmaceutical formulation blend have a volume average diameter of less than 10 μm.

18. The method of claim 1, wherein the pharmaceutical agent has a solubility in water of less than 10 mg/mL at 25° C., wherein the bulking agent comprises at least one sugar, sugar alcohol, starch, amino acid or combination thereof and wherein the non-friable excipient comprises a surfactant.

19. A method for making an oral dosage form of a pharmaceutical agent, comprising the steps of:
a) providing particles which comprise a pharmaceutical agent;
b) blending the particles which comprise a pharmaceutical agent with particles of an excipient to form a first blend;
c) milling the first blend to form a second blend, which comprises microparticles or nanoparticles of the pharmaceutical agent;
d) granulating the second blend to form a granulated milled blend; and
e) processing the granulated milled blend into an oral dosage form.

20. The method of claim 19, wherein the particles of step a) are microparticles.

21. The method of claim 19, wherein the milling of step c) comprises jet milling.

22. The method of claim 19, wherein the granulated milled blend is processed into a solid oral dosage form selected from the group consisting of tablets, capsules, orally disintegrating wafers, and sprinkle packets.

23. The method of claim 19, wherein the granulated milled blend in step e) is processed into a liquid suspension for oral administration.

24. The method of claim 19, wherein step e) comprises:
blending the granulated milled blend with at least one sugar and at least one disintegrant to form a third blend; and
tabletting the third blend to form an orally disintegrating wafer.

25. The method of claim 19, wherein the pharmaceutical agent has a solubility in water of less than 10 mg/mL at 25° C.

26. A method for making a solid oral dosage form of a pharmaceutical agent, comprising the steps of:
a) providing particles which comprise a pharmaceutical agent;
b) blending the particles of pharmaceutical agent with particles of at least one excipient to form a first blend;
c) milling the first blend to form a milled blend which comprises microparticles; and
d) processing the milled blend into a solid oral dosage form,
wherein the size of the microparticles following reconstitution of the solid oral dosage form is not more than 300% of the size of the microparticles in the milled blend pre-processing.

27. The method of claim 26, wherein the size of the microparticles following reconstitution of the solid oral dosage form is not more than 150% of the size of the microparticles in the milled blend pre-processing.

28. The method of claim 26, wherein step d) comprises compacting the milled blend into a unitary dosage form selected from the group consisting of tablets and orally disintegrating wafers.

29. The method of claim 26, wherein the milling of step c) comprises jet milling.

30. The method of claim 26, wherein the pharmaceutical agent has a solubility in water of less than 10 mg/mL at 25° C.

31. The method of claim 26, wherein the microparticles of pharmaceutical agent in the milled blend have a volume average diameter of less than 100 μm.

32. The method of claim 26, wherein the microparticles of pharmaceutical agent in the milled blend have a volume average diameter of less than 10 μm.

33. A method for making a pharmaceutical formulation, comprising the steps of:
a) providing particles which comprise a pharmaceutical agent;
b) blending the particles with particles of a pre-processed excipient to form a primary blend, wherein the pre-processed excipient is prepared by
i) dissolving a bulking agent and at least one non-friable excipient in a solvent to form an excipient solution, and
ii) removing the solvent from the excipient solution to form the pre-processed excipient in dry powder form; and
c) milling the primary blend to form a milled pharmaceutical formulation blend, which comprises microparticles or nanoparticles of the pharmaceutical agent.

34. The method of claim 33, wherein the milling comprises jet milling.

35. The method of claim 33, wherein the bulking agent comprises at least one sugar, sugar alcohol, starch, amino acid, or combination thereof.

36. The method of claim 33, wherein the non-friable excipient comprises a liquid, waxy, or non-crystalline compound.

37. The method of claim 33, wherein the step of removing the solvent comprises spray drying, lyophilization, vacuum drying, or freeze drying.

38. The method of claim 33, wherein the pharmaceutical agent has a solubility in water of less than 10 mg/mL at 25° C.

39. The method of claim 33, wherein the microparticles or nanoparticles of pharmaceutical agent in the milled pharmaceutical formulation blend have a volume average diameter of less than 10 μm.

40. An oral dosage form of a pharmaceutical agent, made by the method of claim 1.

41. An oral dosage form of a pharmaceutical agent, made by the method of claim 19.

42. A solid oral dosage form of a pharmaceutical agent, made by the method of claim 26.

43. A pharmaceutical formulation, made by the method of claim 33.

44. An oral disintegrating tablet comprising:
a mixture of
granules formed by granulation of a milled blend of (i) microparticles which comprise a pharmaceutical agent, and (ii) excipient particles;
particles of at least one sugar; and
particles of at least one disintegrant,
wherein the mixture has been compressed into a tablet or wafer form.

45. The oral disintegrating tablet of claim 44, wherein the pharmaceutical agent has a solubility in water of less than 10 mg/mL at 25° C.

46. The oral disintegrating tablet of claim 44, wherein the excipient particles comprise a hydrophilic surfactant.

47. A solid oral dosage form of a pharmaceutical agent, comprising:
a milled blend of microparticles of a pharmaceutical agent blended and particles of at least one excipient, which milled blend has been processed into a solid oral dosage form,
wherein the size of the microparticles following reconstitution of the solid oral dosage form is not more than 300% of the size of the microparticles in the milled blend pre-processing.

48. The solid oral dosage form of claim 47, wherein the size of the microparticles following reconstitution of the solid oral dosage form is not more than 200% of the size of the microparticles in the milled blend pre-processing.
